SEQUENCE LISTING (12 sequences)

SEQ ID NO: 1 - protein, 167 aa, Corynebacterium glutamicum ATCC 13032
  PEPTIDE (1)..(167): NCgl2399 gluconate kinase
MSAAEGLHIVVMGVSGCGKSSVGKALAAELGIEYKDGDELHPQENIDKMASGQALDDDDRAWWLVQVGKWLRDRPSGVIACSALKRSYRDLLRTKCPGTVFVHLHGDYDLLLSRMKAREDHFMPSTLLDSQFATLEPLEDDEDGKVFDVAHTISELAAQSAEWVRNK

SEQ ID NO: 2 - protein, 494 aa, Corynebacterium glutamicum ATCC 13032
  PEPTIDE (1)..(494): NCgl2905 gluconate kinase
MGSIPTMSIPFDDSRGPYVLAMDIGSTASRGGLYDASGCPIKGTKQRESHEFTTGEGVSTIDADQVVSEITSVINGILNAADHHNIKDQIAAVALDSFASSLILVDGEGNALTPCITYADSRSAQYVEQLRAEIDEEAYHGRTGVCLHTSYHPSRLLWLKTEFEEEFNKAKYVMTIGEYVYFKLAGITGMATSIAAWSGILDAHTGELDLTILEHIGVDPALFGEIRNPDEPATDAKVVDKKWKHLEEIPWFHAIPDGWPSNIGPGAVDSKTVAVAAATSGAMRVILPSVPEQIPSGLWCYRVSRDQCIVGGALNDVGRAVTWLERTIIKPENLDEVLIREPLEGTPAVLPFFSGERSIGWAASAQATITNIQEQTGPEHLWRGVFEALALSYQRVWEHMGKAGAAPERVIASGRVSTDHPEFLAMLSDALDTPVIPLEMKRATLRGTALIVLEQLEPGGTRATPPFGTTHQPRFAHHYSKARELFDALYLKLV

SEQ ID NO: 3 - DNA, 504 bp, Corynebacterium glutamicum ATCC 13032
  gene (1)..(504): nucleotide sequence of NCgl2399 gluconate kinase
atgtcagcagccgaaggcttacatattgtcgtcatgggcgtttctggctgcggcaaatcctccgtcggtaaagccctagcagcggagctcggaatcgaatacaaagacggcgacgaacttcacccccaggaaaacatcgacaagatggcctccggccaggcacttgacgacgacgaccgtgcatggtggctagtccaggttggcaagtggctccgcgaccgaccaagcggcgtcatcgcatgctccgccctcaagcgctcctaccgcgatctcctgcgcaccaaatgcccaggaaccgtcttcgtccacctccacggcgactacgatctcctactttcccgcatgaaggcccgcgaagatcacttcatgccatccaccttgctagattcccaatttgcaaccctcgagccgctcgaagatgacgaagatggcaaggttttcgacgttgcccacaccatcagcgaactggccgcccaatctgcagagtgggttcgcaacaaataa

SEQ ID NO: 4 - DNA, 1485 bp, Corynebacterium glutamicum ATCC 13032
  gene (1)..(1485): nucleotide sequence of NCgl2905 gluconate kinase
atgggatcaattccaacaatgtccatcccttttgatgactcacgtggaccttatgtccttgctatggatattggttccactgcatcacgaggtggactttatgatgcttccggctgcccaatcaaaggcaccaagcagcgcgaatcccatgaattcaccaccggtgagggcgtttccaccattgatgctgaccaggtggtttcggagatcacctcagttattaatggcattttgaacgcggctgatcatcacaacatcaaagatcagatcgccgctgtcgcgctagattcttttgcatcctcattaatcttggtcgatggtgaaggcaatgcgctcaccccgtgcattacctacgcggattctcgttctgcacagtatgtggagcagctgcgcgcggaaatcgatgaggaggcctaccacggccgcaccggcgtctgcctgcacacctcctaccacccatcgcgcctgctgtggctgaaaactgagttcgaggaagagttcaacaaagccaagtatgtgatgaccatcggtgagtacgtctacttcaaacttgcaggcatcaccggaatggctacttcgattgccgcgtggagtggcattttggacgcccataccggcgaacttgatctgactatcttggagcacatcggtgttgatccggctctgttcggtgagatcagaaaccctgatgaaccagccaccgatgccaaagttgtcgacaaaaagtggaagcacctggaagaaatcccttggttccatgccattccagacggctggccttccaacattggcccaggcgccgtggattctaaaaccgtcgcagtcgccgccgctacatccggcgccatgcgcgtgatccttccgagcgttcccgaacagatcccctctggcctgtggtgttaccgcgtttcccgcgaccagtgcatcgttggtggcgcactcaacgacgtcggacgcgccgtcacctggctggaacgcaccattatcaagcctgaaaacctcgacgaagtgctgatccgcgaacccctcgaaggcaccccagctgtcctgccgttcttctccggggaacgctccatcggctgggcagcctcagcgcaggccacgatcaccaacattcaggaacaaaccggccctgaacacttgtggcgcggcgttttcgaagccctcgcactctcctaccagcgcgtttgggaacacatggggaaagccggcgcagcccctgaacgggtcatcgcatcaggacgagtctccaccgaccacccagaattcctcgcgatgctttccgacgccctcgacaccccagtcatccctctggaaatgaagcgcgccaccctccgcggcaccgcacttatcgtccttgagcagctcgaaccaggcggcacgcgcgcgacgccaccattcggcacgacgcatcagccgcgctttgcgcaccattactccaaggcaagagagcttttcgacgccctctacctcaagttggtctag

SEQ ID NO: 5 - DNA, 26 bp, Artificial Sequence
  primer 2399F1
gctctagaccccagaacatgctgacg

SEQ ID NO: 6 - DNA, 26 bp, Artificial Sequence
  primer 2399R1
ggggtacccgctgctagggctttacc

SEQ ID NO: 7 - DNA, 27 bp, Artificial Sequence
  primer 2399F2
ggggtaccggaaccgtcttcgtccacc

SEQ ID NO: 8 - DNA, 26 bp, Artificial Sequence
  primer 2399R2
gctctagagtcaccgctggtacatcc

SEQ ID NO: 9 - DNA, 26 bp, Artificial Sequence
  primer 2905F1
gctctagacattggagcatccgtagc

SEQ ID NO: 10 - DNA, 27 bp, Artificial Sequence
  primer 2905R1
tcccccggggccttcaccatcgaccaa

SEQ ID NO: 11 - DNA, 27 bp, Artificial Sequence
  primer 2905F2
tcccccggggttcccgaacagatcccc

SEQ ID NO: 12 - DNA, 26 bp, Artificial Sequence
  primer 2905R2
gctctagacgctctgacctgcctaac

(19) United States
(12) Patent Application Publication
(10) Pub. No.: US 2013/0102037 A1
(43) Pub. Date: Apr. 25, 2013

(54) Microorganism with Enhanced L-Lysine Productivity and Method for Producing L-Lysine Using the Same

(71) Applicants: Sang Jo Lim, Incheon (KR); Jun Ok Moon, Seoul (KR); Hyung Joon Kim, Seoul (KR); Jae Woo Jang, Gyeonggi-do (KR); Jae Yong Cho, Gangwon-do (KR)

(72) Inventors: Sang Jo Lim, Incheon (KR); Jun Ok Moon, Seoul (KR); Hyung Joon Kim, Seoul (KR); Jae Woo Jang, Gyeonggi-do (KR); Jae Yong Cho, Gangwon-do (KR)

(73) Assignee: CJ CHEILJEDANG CORPORATION, Seoul (KR)

(21) Appl. No.: 13/378,145

(22) PCT Filed: Jul. 15, 2011
(86) PCT No.: PCT/KR11/05252

§ 371 Date: Dec. 14, 2011

(30) Foreign Application Priority Data
Jul. 15, 2010 (KR) 10-2010-0068618
Jul. 1, 2011 (KR) 10-2011-0065694

(51) Int. Cl. C12N 15/77

(52) U.S. Cl. CPC C12N 15/77
USPC 435/115; 435/252.32; 435/487

(57) ABSTRACT

Disclosed is an L-lysine-producing microorganism having gluconate kinase activity weakened in comparison to the endogenous activity thereof, and methods provided for preparing the microorganism and for producing L-lysine using the same.

### TECHNICAL FIELD

The present invention relates to a microorganism with high L-lysine productivity and a method for producing L-lysine using the same.

### BACKGROUND ART

L-Lysine is synthesized from oxaloacetic acid via the lysine biosynthetic pathway. The enzymes important to the biosynthesis of L-lysine are aspartate semialdehyde dehydrogenase, dihydrodipicolinate reductase and diaminopimelate dehydrogenase, which are respectively encoded by the genes asd, dapB and ddh, and which are NADPH-dependent reductases that mediate parts of the lysine biosynthetic pathway. In this pathway, the production of one molecule of L-lysine directly requires the consumption of three molecules of NADPH by the enzymes and one molecule of NADPH indirectly. Direct correlation between the regeneration of NADPH and the biosynthesis of L-lysine in the Corynebacterium glutamicum cell was previously reported (Wittmann and Heinzle, Microbiol 68:5843-5849, 2002; Marx et al., J Biotechnol 104:185-197, 2003; Ohnishi et al., Microbiol Lett 242:265-274, 2005).

In Corynebacterium, the main supply routes of NADPH are the TCA cycle and the pentose phosphate pathway. It is preferred that the reducing power necessary for the production of L-lysine be supplied via oxidation pathway of the pentose phosphate pathway. The pentose phosphate pathway is economically more beneficial in terms of carbon metabolism yield because the operation of two cycles of the pentose phosphate pathway releases one CO₂ molecule with the concomitant production of two NADPH molecules whereas two CO₂ molecules are released per one TCA cycle, with the concomitant production of one NADPH molecule. Thus, a higher yield of L-lysine requires a greater supply of NADPH, resulting in a higher dependence on the pentose phosphate pathway.

Glucose-6-phosphate dehydrogenase (G6PDH) and 6-phosphogluconate dehydrogenase (6PGD), both involved in the pentose phosphate pathway, were reported to be associated with the production of L-lysine (Marx et al., Biotechnol Bioeng 56:168-180, 1997; Wittmann and Heinzle, Microbiol 68:5843-5849, 2002).

In L-amino acid-producing Corynebacterium strains, when genes encoding enzymes responsible for the production of NADPH in the pentose phosphate pathway are enhanced or when the enzymes are mutated, L-amino acid productivity is reported to be increased. For example, J. Becker et al., reported that the provision of a stronger promoter for the zwf gene encoding G6PDH in L-lysine-producing C. glutamicum brought about a significant increase in lysine production (J. Becker et al., J Biotechnol 132: 99-109, 2007). European Patent No. 1302537 discloses a method for producing L-amino acids by culturing L-amino acid-producing corynebacteria into a gene encoding a mutant of 6PGD which has been introduced.

A Corynebacterium sp. microorganism in which the NADPH biosynthesis in the pentose phosphate pathway was increased by attenuating the endogenous gluconate kinase (GntK) activity of Corynebacterium, however, has not been disclosed in previous reports.

### DISCLOSURE OF INVENTION

### Technical Problem

Leading to the present invention, intensive and thorough research, conducted by the present inventors, aimed at finding a method for production of L-lysine at high efficiency and yield, resulted in the finding that an increase in the production of NADPH by genetic modification in the oxidative pentose phosphate pathway can increase yield of L-lysine.

### Solution to Problem

It is therefore an object of the present invention to provide an L-lysine-producing microorganism that has gluconate kinase (GntK) activity weakened in comparison with the endogenous activity.

It is another object of the present invention to provide a method for producing the L-lysine-producing microorganism, comprising: constructing a polynucleotide fragment encoding gluconate kinase (GntK) having a weakened activity by wholly or partially mutating said polynucleotide; inserting the polynucleotide fragment into a vector to afford a recombinant vector, said vector being capable of homologous recombination with a chromosome in a host cell; introducing the recombinant vector into a host cell capable of producing L-lysine to form homologous recombinants; and selecting a strain having GntK activity weakened in comparison to the endogenous activity thereof, from the homologous recombinants.

It is a further object of the present invention to provide a method for producing L-lysine, comprising: culturing the microorganism of the present invention to obtain a cell culture; and harvesting L-lysine from the cell culture or the microorganism.

### Advantageous Effects of Invention

The present invention can produce L-amino acids in need of NADPH for their biosynthesis at high efficiency and yield, especially L-lysine, via increasing the intracellular level of NADPH in Corynebacterium strains.

### BRIEF DESCRIPTION OF DRAWINGS

The above and other objects, features and other advantages of the present invention will be more clearly understood from the following detailed description taken in conjunction with the accompanying drawings, in which:

FIG. 1 is a schematic diagram illustrating the pentose phosphate pathway in Corynebacterium glutamicum;

FIG. 2 is a schematic diagram illustrating the pDZ-ΔNCgl2399 vector for marker-free deletion of the chromosomal NCgl2399 gene of Corynebacterium; and

FIG. 3 is a schematic diagram illustrating the pDZ-ΔNCgl2905 vector for marker-free deletion of the chromosomal NCgl2905 gene of Corynebacterium.

### BEST MODE FOR CARRYING OUT THE INVENTION

In accordance with an aspect thereof, the present invention provides an L-lysine-producing microorganism that has a gluconate kinase (GntK) activity weakened in comparison with the endogenous activity.

L-lysine is a basic a-amino acid with the chemical formula of NH₂(CH₂)₄CH(NH₂) COOH. It is an essential amino acid, which means that the human body cannot synthesize it. L-lysine is synthesized from oxaloacetic acid via the lysine biosynthesis pathway a part of which is catalyzed by NADPH-dependent reductases. The biosynthesis of one L-lysine molecule via this pathway requires the direct consumption of three NADPH molecules by enzymes, with the indirect use of one NADPH molecule.

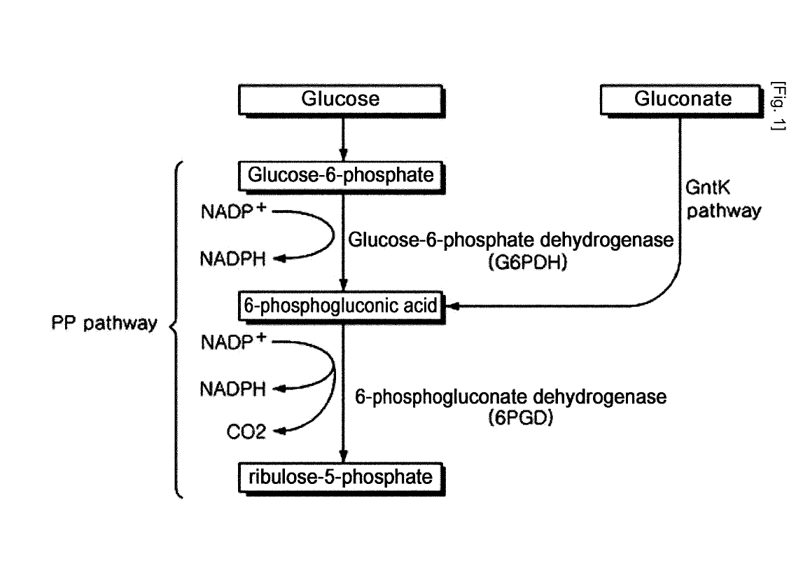

The term “gluconate kinase (GntK),”as used herein, refers to an enzyme that is concerned in the GntK pathway which produces 6-phosphogluconic acid, an intermediate in oxidative process of the pentose phosphate pathway in microorganisms. Microorganisms of Corynebacterium spp. operate both pathways, that is, the G6PDH and GntK pathways, to synthesize 6-phosphogluconic acid, an intermediate of the pentose phosphate pathway (FIG. 1), which means that microorganisms of Corynebacterium spp. may partially degrade glucose via the GntK pathway. On the presumption that the blockage of the GntK pathway may interrupt the provision of carbon to 6-phosphogluconic acid and convert carbon flux from 6-phosphogluconic acid to the oxidation process of the pentose phosphate pathway, the present inventors suggest that the blockage of the GntK pathway gives rise to a specific increase in the activity of 6-phosphogluconate dehydrogenase (6PGD), thus increasing the regeneration of NADPH. In this context, the activity of the enzyme which is presumed to act as gluconate kinase (GntK) in microorganisms is weakened.

Preferably, the enzyme having gluconate kinase (GntK) activity may be NCgl2399 represented by the amino acid sequence of SEQ ID NO: 1 or NCgl2905 represented by the amino acid sequence of SEQ ID NO: 2.

As used herein, the term “endogenous activity” is intended to mean the activity of an enzyme in a native microorganism, and particularly when used in combination with GntK, refers to the activity of GntK that a native microorganism exhibits.

The term “weakness of endogenous activity” is achieved on a chromosome containing a polynucleotide encoding an endogenous protein in a microorganism, by a method selected from the group consisting of a whole or partial deletion of the polynucleotide sequence, partial substitution of the polynucleotide sequence, or at least one base pair insertion into the polynucleotide sequence. In addition, endogenous activity can be weakened by mutating a regulatory element for the polynucleotide sequence, where the mutation is achieved by whole or partial deletion, substitution, or insertion of the regulatory element. The regulatory element may be on the upstream or downstream of the polynucleotide sequence in a chromosome, preferably, the regulatory element may be a promoter, enhancer et, al, but the present invention is not limited thereto. The mutation of the polynucleotide may be achieved using a well-known method, such as a homologous recombination.

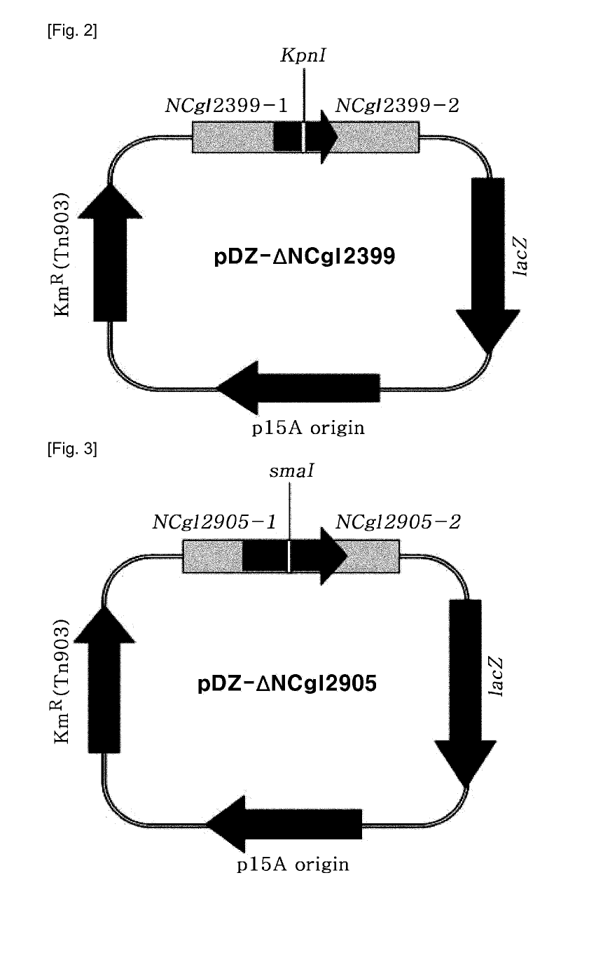

The term “GntK activity weakened in comparison to the endogenous activity,” as used herein, is intended to mean that GntK activity is down-regulated by genetic mutation such as disruption and thus becomes lower than the endogenous activity of the enzyme in a native microorganism. In an embodiment of the present invention, the present invention provides microorganisms of Corynebacterium spp. with enhanced L-lysine productivity by disrupting GntK activity via introducing a recombinant vector represented by the cleavage map in FIG. 2 or 3.

When there are two or more chromosomal genes coding proteins having gluconate kinase activity and having different amino acid sequences to each other, the endogenous GntK activity can be weakened by whole or partial deletion, substitution or insertion on at least one of the chromosomal genes. And endogenous GntK activity can be weakened by whole or partial deletion, partial substitution or insertion on a regulatory element for at least one of the chromosomal genes.

Preferably, a mutation by deletion, substitution or insertion of nucleotides may be allowed to occur on a gene encoding NCgl2399 or NCgl2905, or either one or both of the genes having nucleotide sequences of SEQ ID NOS: 3 and 4 which code respectively for NCgl2399 and NCgl2905, or on regulatory elements thereof, so that GntK does not function normally.

In the present invention, a microorganism with gluconate kinase activity weakened in comparison with the endogenous activity thereof can be prepared by introducing a recombinant vector carrying a part of the gene encoding gluconate kinase thereinto to delete or mutate the endogenous gluconate kinase gene thereof. The insertion of the partial gene into the chromosome may be achieved using a well-known method, such as a homologous recombination.

In this regard, the present invention provides a method for preparing an L-lysine-producing microorganism, comprising the steps of: 1) constructing a polynucleotide fragment encoding gluconate kinase (GntK) having a weakened activity by wholly or partially mutating said polynucleotide; 2) inserting the polynucleotide fragment into a vector to afford a recombinant vector, said vector being capable of a homologous recombination with a chromosome in a host cell; 3) introducing the recombinant vector into a host cell capable of producing L-lysine to form homologous recombinants; and 4) selecting a strain having GntK activity attenuated in comparison to the endogenous activity thereof, from the homologous recombinants.

The term “recombinant vector,” as used herein, is intended to refer to a vector that cannot be replicated separately from the chromosome of a host cell and typically means a genetic construction comprising essential elements which allow the homologous recombination of a foreign gene with the chromosome. The recombinant vector may carry an antibiotic-resistance gene and a selection gene such as levansucrase (sacB) gene. Preferably, the recombinant vectors represented by the cleavage maps of FIGS. 2 and 3 may be employed in the present invention.

As long as L-lysine can be produced, any strain may be used without limitation as a host cell into which the recombinant vector is introduced. Preferred are the microorganisms of Corynebacterium spp. or Brevibacterium spp. Examples of host cells useful in the present invention include C. glutamicum ATCC13032, C. thermoaminogenes FERM BP-1539, Brevibacterium flavum ATCC 14067, and Brevibacterium lactofermentum ATCC 13869, and L-amino acid-producing mutants or stains derived therefrom, such as C. glutamicum KFCC10881, C. glutamicum KFCC11001 and C. glutamicum KCCM10770P. Preferred is C. glutamicum KFCC10881. In an embodiment of the present invention, C. glutamicum KFCC10881 was employed, but the present invention is not limited thereto.

In an embodiment of the present invention, the pDZ vector (disclosed in Korean Patent No. 0924065), which can perform the marker-free deletion of a target gene in C. glutamicum without replication, was utilized to delete the genes. That is, a recombinant vector carrying a part of the gene encoding NCgl2399 or NCgl2905 was constructed, transformed into an L-lysine-producing Corynebacterium strain, and allowed to perform homologous recombination with the genome to prepare L-lysine-producing Corynebacterium strain having weakened gluconate kinase activity.

In the present invention, a part of a gene encoding NCgl2399 was inserted into a pDZ vector to construct a recombinant vector, called pDZ-ΔNCgl2399, which was then transformed into a Corynebacterium strain. The resulting recombinant Corynebacterium strain in which the chromosomal gene encoding NCgl2399 was disrupted was named KFCC10881-ΔNCgl2399. Separately, a recombinant pDZ vector carrying a part of a gene encoding NCgl2905, named pDZ-ΔNCgl2905, was transformed into a Corynebacterium strain. The resulting recombinant strain in which the chromosomal gene encoding NCgl2905 was disrupted was named KFCC10881-ΔNCgl2905. Further, KFCC10881-ΔNCgl2399 was transformed with the recombinant plasmid pDZ-ΔNCgl2905 to provide a mutant strain which was defective in both NCgl2399 and NCgl2905 genes. The strain was named C. glutamicum CA01-0892 and deposited with the Korean Culture Center of Microorganisms on Jun. 24, 2010, under accession No. KCCM 11085P.

Further, in one embodiment of the present invention, the recombinant C. glutamicum strains were evaluated for intracellular gluconate kinase activity and NADPH level. The intracellular GntK activity of the strain which is defective in the NCgl2399 gene and the strain which is defective in both the NCgl2399 and NCgl2905 gene were observed to have been decreased compared to the parent strain. In addition, intracellular NADPH level and 6PGD activity were observed to have been increased compared to the parent strain (Table 2). Consequentially, L-lysine production yield of the prepared strains were observed to have been increased compared to the parent strain (Table 3).

These results demonstrate that the attenuation of gluconate kinase activity below the endogenous activity increases 6PGD activity, which has a positive effect on the generation of NADPH, thus resulting in the production of L-lysine at high efficiency and yield. In addition, in the case where a protein having gluconate kinase activity is encoded by two or more different genes, higher L-lysine productivity can be obtained by deleting two or more of the genes. Therefore, the L-lysine-producing microorganisms of the present invention can be used for producing L-lysine at high efficiency and yield.

In accordance with another aspect thereof, the present invention provides a method for producing L-lysine, comprising the steps of: 1) culturing the microorganism of the present invention to obtain a cell culture; and 2)havesting L-lysine from the cell culture or the microorganism.

As used herein, the term “culturing” means allowing microorganisms to grow under artificially controlled conditions. Various methods well known in the art may be employed to culture the recombinants of the present invention. The cells may be cultured in a batch process or in a continuous process such as a fed-batch process or a repeated fed batch process, but the present invention is not limited thereto.

For use in the culturing, a medium must satisfy the requirement of the strain employed. Culture media suitable for use in culturing Corynebacteria strains are well known in the art (e.g., Manual of Methods for General Bacteriology, American Society for Bacteriology, Washington D.C., USA, 1981). Carbon sources of the culture media may be saccharides and carbohydrates such as glucose, sucrose, lactose, fructose, maltose, starch and cellulose; oils and lipids such as soybean oil, sunflower seed oil, peanut oil and coconut oil; fatty acids such as palmeatic acid, stearic acid, rinoleic acid;, alcohols such as glycerol and ethanol; and organic acids such as acetic acid. These materials may be used separately or in combination. Examples of a nitrogen source useful for culturing the recombonants include peptone, yeast extract, broth, malt extract, corn steep liquor, soybean meal and urea, or inorganic compounds such as ammonium sulfate, ammonium chloride, ammonium phosphate, ammonium carbonate and ammonium nitrate. These nitrogen sources may be used separately or in combination. Among phosphorus sources useful in the culture media are dipotassium hydrogen phosphate, potassium dihydrogen phosphate and corresponding sodium salts. In addition, culture media may contain metal salts essential to the growth of cells and may be supplemented with essential nutrients such as amino acids and vitamins In addition, proper precursors may be added to the culture media. The nutrients or supplements may be added altogether once or in separation during cultivation.

The pH of the culture media may be adjusted with a basic compound such as sodium hydroxide, potassium hydroxide or ammonia or an acidic compound such as phosphoric acid or sulfuric acid. The generation of foam in the culture media may be restrained using an anti-foaming agent such as fatty acid polyglycol ester. The culture media may be kept under aerobic conditions by introducing oxygen or an oxygen-containing gas (e.g., air) thereinto. The culture temperature is typically between 20 and 45° C. and preferably between 25 and 40° C. The culturing is continued until a maximal amount of L-amino acid is produced. In this regard, it may be accomplished within 10 to 160 hrs. After being produced, the L-lysine may be exported into the culture media or may remain within the cells.

The method for the production of L-lysine in accordance with the present invention comprises harvesting lysine from the cells or the cell culture. L-lysine can be isolated from culture media or cells using a well-known method. Examples of the isolation method useful in the present invention include centrifugation, filtration, anionic exchange chromatography, crystallization and HPLC, but are not limited thereto.

### MODE FOR THE INVENTION

A better understanding of the present invention may be obtained through the following examples which are set forth to illustrate, but are not to be construed as limiting the present invention.

### EXAMPLE 1

### Preparation of Mutant Strains by Site-Specific Gene Disruption

For use in the preparation of the recombinant strains KFCC10881-ΔNCgl2399 and KFCC10881-ΔNCgl2905, primers were designed. To begin with, sequences of NCgl2399(SEQ ID NO: 1) and NCgl2905(SEQ ID NO: 2) were obtained from the NIH GenBank. On the basis of these sequences, the primers 2399F1, 2399F2, 2399R1, 2399R2, 2905F1, 2905F2, 2905R1, and 2905R2 (Table 1) were synthesized which would be used in constructing inactivation fragments of NCgl2399 or NCgl2905. Nucleotide sequences of the primers used in the present invention, along with their SEQ ID NOs., are summarized in Table 1 wherein restriction sites are underlined.

TABLE 1

| Primer | Nucleotide Sequence | SEQ ID No: |
|---|---|---|
| 2399F1 | gctctagaCCCCAGAACATGCTGACG(XbaI) | 5 |
| 2399R1 | ggggtaccCGCTGCTAGGGCTTTACC(KpnI) | 6 |
| 2399F2 | ggggtaccGGAACCGTCTTCGTCCACC(KpnI) | 7 |
| 2399R2 | gctctagaGTCACCGCTGGTACATCC(XbaI) | 8 |
| 2905F1 | gctctagaCATTGGAGCATCCGTAGC(XbaI) | 9 |
| 2905R1 | tcccccgggGCCTTCACCATCGACCAA(SmaI) | 10 |
| 2905F2 | tcccccgggGTTCCCGAACAGATCCCC(SmaI) | 11 |
| 2905R2 | gctctagaCGCTCTGACCTGCCTAAC(XbaI) | 12 |

Site-specific gene disruption was conducted with pDZ, which is incapable of replication in C. glutamicum. For use in preparing gene-disrupted mutant strains, pDZ derivatives carrying respective internally defective open reading frames of NCgl2399 and NCgl2905 were constructed. The pDZ derivatives are pDZ-ΔNCgl2399 (FIG. 2) and pDZ-ΔNCgl2905 (FIG. 3). pDZ-ΔNCgl2399 carries an XbaI end and an internal gene defective at a KpnI site of a 2,267 bp-long NCgl2399. The internal gene defective of NCgl2399 was generated using overlap extension PCR in the presence of 2399F1-2399R1 primers (SEQ ID NOS: 5 and 6) and 2399F2-2399R2 primers (SEQ ID NOS: 7 and 8), with the genomic DNA of C. glutamicum ATCC13032 serving as a template.

pDZ-ΔNCgl2905 carries a XbaI end and an internal gene defective at a SmaI site of a 2,819 bp-long NCgl2905. The internal gene defective of NCgl2905 was generated using overlap extension PCR in the presence of 2905F1-2905R1 primers (SEQ ID NOS: 9 and 10) and 2905F2-2905R2 primers (SEQ ID NOS: 11 and 12), with the genomic DNA of C. glutamicum ATCC13032 serving as a template.

The recombinant plasmids were transformed into wild-type Corynebacterium glutamicum using electroporation (van der Rest et al., Appl Microbiol Biotechnol 52:541-545, 1999) and incorporated into the chromosome by primary recombination (crossover). Then, the plasmids were excised from the chromosomes by secondary recombination (crossover) on an agar plate containing 10% sucrose.

Using gene-specific primer pairs 2399F1-2399R2 (SEQ ID NOS: 5 and 8) and 2905F1-2905R2 (SEQ ID NOS: 9 and 12), diagnostic PCR was performed on the genomic DNA of the C. glutamicum recombinants in which the secondary recombination was completed, confirming that the C. glutamicum recombinants had defective NCgl2399 and NCgl2905 genes, respectively. These recombinant strains were named KFCC10881-ΔNCgl2399 and KFCC10881-ΔNCgl2905, respectively. The open reading frame DNA sequences of NCgl2399 and NCgl2905 are referred to GenBank Accession No. NC_{—}003450.

### EXAMPLE 2

### Prepration of Mutant Strain Defective in Both NCgl2399 and NCgl2905 Genes

The recombinant strain KFCC10881-ΔNCgl2399 was transformed with the recombinant plasmid pDZ-ΔNCgl2905 using electroporation and treated in the same manner as illustrated above to give a novel recombinant strain that were defective in both NCgl2399 and NCgl2905 genes. This mutant strain was named C. glutamicum CA01-0892 and deposited with the Korean Culture Center of Microorganisms on Jun. 24, 2010, under accession No. KCCM 11085P.

### EXAMPLE 3

### Assay for Intracellular GntK and 6PGD Activity and NADPH Level

The L-lysine-producing strains C. glutamicum KFCC-10881-ΔNCgl2399, KFCC-10881-ΔNCgl2905 and CA01-0892 (KFCC-10881-ΔNCgl2399ΔNCgl2905), prepared in Examples 1 and 2, were assayed for intracellular gluconate kinase activity and the NADPH levels are as follows.

The parent strain C. glutamicum KFCC-10881 and the three recombinant strains were inoculated into respective 250 mL corner-baffle flasks containing 25 mL of the following complex medium and cultured at 30° C. with shaking at 200 rpm. The cells in the exponential phase were harvested by centrifugation and suspended in 100 mM Tris/HCl buffer (pH 7.5). The cells were disrupted using glass beads, followed by centrifuging the cell lysate to obtain a supernatant.

<Complex Medium (pH 7.0)>

Glucose 20 g, Peptone 10 g, Yeast Extract 5 g, Urea 1.5 g, KH₂PO₄ 4 g, K₂HPO₄ 8 g, MgSO₄ 7H₂O 0.5 g, Biotin 100 μg, Thiamine HCl 1000 μg, Calcium-pantothenate 2000 μg, Nicotine amide 2000 μg (in 1 liter of distilled water)

The supernatants were quantitatively analyzed for total protein contents by a Bradford method. 6PGD activity was determined using absorbance at 340 nm for NADPH (Frunzke et al., Mol Microbiol 67:305-322, 2008). GntK activity was measured by the coupled enzymatic assay for 6PGD (Frunzke et al., Mol Microbiol 67:305-322, 2008). NADPH levels were determined by an enzymatic cycling reaction with the aid of EnzyChrom™ NADP⁺/NADPH assay kit (BioAssay Systems, CA, USA).

As can be seen in Table 2, the intracellular GntK (EC:2.7.1.12) activity of KFCC-10881-ΔNCgl2399, which was defective in the NCgl2399 gene, was decreased by about 58.7% but there was a 2.4-fold increase in 6PGD activity, compared to the parent strain (KFCC10881). GntK activity of CA01-0892, defective in both NCgl2399 and NCgl2905 gene, was decreased by about 78.3%, but there was a 5.1-fold increase in 6PGD activity, compared to the parent strain (Table 2).

As a result of the enzymatic activity change, it was also observed that the intracellular NADPH level CA01-0892 was increased by up to 170%. These data were averages of measurements from at least three independent cultures, with standard deviations less than 10% in all cases. The results indicate that the attenuation of gluconate kinase activity below the endogenous activity increases 6PGD activity, which has a positive effect on the generation of NADPH.

TABLE 2

Enzyme
Specific activity
(U mg protein⁻¹)

| Strain | GntK | 6PGD |
|---|---|---|
| KFCC-10881 | 0.46 | 0.99 |
| KFCC-10881-ΔNCgl2399 | 0.19 | 2.4 |
| CA01-0892 | 0.10 | 5.1 |
| (KFCC-10881-ΔNCgl2399ΔNCgl2905) | | |

### EXAMPLE 4

### Production of Lysine in Strains Defective in NCgl2399 and NCgl2905 Genes

To produce L-lysine, the L-lysine-producing strains C. glutamicum KFCC-10881-ΔNCgl2399, KFCC-10881-ΔNCgl2905 and CA01-0892 (KFCC-10881-ΔNCgl2399ΔNCgl2905), prepared in Examples 1 and 2, were cultured as follows.

The parent strain C. glutamicum KFCC-10881, and the three recombinant strains were inoculated into 250 mL-corner baffle flasks containing 25 mL of the following seed medium and cultured at 30° C. for 20 hours with shaking at 200 rpm. Thereafter, 1 mL of each of the cultures were inoculated into a 250 mL-corner baffle flask containing 24 mL of the following production medium and cultured at 30° C. for 120 hours with shaking at 200 rpm. The seed medium and the production medium comprise the following compositions.

<Seed Medium (pH 7.0)>

Glucose 20 g, peptone 10 g, yeast extract 5 g, urea 1.5 g, KH₂PO₄ 4 g, K₂HPO₄ 8 g, MgSO₄ 7H₂O 0.5 g, biotin 100 μg, thiamine HCl 1000 μg, calcium-pantothenate 2000 μg, nicotinamide 2000 μg (in 1 liter of distilled water)

<Production Medium (pH 7.0)>glucose 100 g, (NH₄)₂SO₄ 40 g, soy protein 2.5 g, corn steep solids 5 g, urea 3 g, KH_{2 PO}₄ 1 g, MgSO₄ 7H₂O 0.5 g, biotin 100 μg, thiamine chloride 1000 μg, calcium-pantothenate 2000 μg, nicotinamide 3000 μg, and CaCO₃ 30 g (in 1 liter of distilled water).

After the completion of culture, HPLC analysis was performed to determine the amounts of the L-lysine produced by the strains. The concentrations of L-lysine in the cultures of C. glutamicum KFCC-10881, KFCC-10881-ΔNCgl2399, KFCC-10881-ΔNCgl2905 and CA01-0892(KFCC-10881-ΔNCgl2399ΔNCgl2905) are summarized in Table 3, below.

TABLE 3

| | Lysine (g/l) | | |
|---|---|---|---|
| Strain | Batch1 | Batch2 | Batch3 |
| KFCC-10881 | 42.1 | 42.6 | 42.4 |
| KFCC-10881-ΔNCgl2399 | 44.9 | 45.5 | 45.2 |
| KFCC-10881-ΔNCgl2905 | 46.0 | 46.2 | 46.2 |
| CA01-0892 (KFCC-10881- | 48.1 | 48.7 | 48.3 |
| ΔNCgl2399ΔNCgl2905) | | | |

As can be seen in Table 3, the recombinant strains defective in the NCgl2399 or NCgl2905 gene, were found to increase in lysine productivity by about 5.9% and 8.0%, respectively, compared with the parent strain KFCC-10881. In addition, it was measured that an increase of about 13.1% in lysine production was obtained with CA01-0892, defective in both NCgl2399 and NCgl2905 genes, in comparison to the parent strain (Table 3). These results demonstrate that the attenuation of gluconate kinase activity the endogenous activity increases 6PGD activity, which has a positive effect on the generation of NADPH, thus resulting in the production of L-lysine at high efficiency and yield.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An L-lysine-producing microorganism, having gluconate kinase (GntK) activity weakened in comparison with the endogenous activity thereof.

2. The L-lysine-producing microorganism of claim 1, wherein the gluconate kinase has an amino acid sequence of SEQ ID NO: 1 or SEQ ID NO: 2.

3. The L-lysine-producing microorganism of claim 1, wherein the microorganism belongs to a Corynebacterium sp.

4. The L-lysine-producing microorganism of claim 1, wherein the endogenous gluconate kinase activity is weakened by whole or partial deletion, partial substitution or insertion on a chromosomal gene encoding gluconate kinase.

5. The L-lysine-producing microorganism of claim 1, wherein the endogenous gluconate kinase activity is weakened by whole or partial deletion, partial substitution or insertion on a regulatory element for a chromosomal gene encoding gluconate kinase.

6. The L-lysine-producing microorganism of claim 1, wherein when there are two or more chromosomal genes coding proteins having gluconate kinase activity and having different amino acid sequences to each other, the endogenous gluconate kinase activity is weakened by whole or partial deletion, substitution or insertion on at least one of the chromosomal genes; or whole or partial deletion, partial substitution or insertion on a regulatory element for at least one of the chromosomal genes.

7. The L-lysine-producing microorganism of claim 1, which is derived from Corynebacterium glutamicum KFCC10881.

8. The L-lysine-producing microorganism of claim 1, identified as Corynebacterium glutamicum CA01-0892 deposited under accession No. KCCM 11085P.

9. A method for preparing the L-lysine-producing microorganism according to claims 1, comprising the steps of:
1) constructing a polynucleotide fragment encoding gluconate kinase (GntK) having a weakened activity by wholly or partially mutating said polynucleotide;
2) inserting the polynucleotide fragment into a vector to afford a recombinant vector, said vector being capable of homologous recombination with a chromosome in a host cell;
3) introducing the recombinant vector into a host cell capable of producing L-lysine to form homologous recombinants; and
4) selecting a strain having a GntK activity weakened in comparison to the endogenous activity thereof, from the homologous recombinants.

10. A method for producing L-lysine, comprising the steps of:
1) culturing a microorganism to obtain a cell culture, wherein the microorganism has gluconate kinase (GntK) activity weakened in comparison with the endogenous activity thereof; and
2) harvesting L-lysine from the cell culture or the microorganism.

11. The method according to claim 10, wherein the gluconate kinase has an amino acid sequence of SEQ ID NO: 1 or SEQ ID NO: 2.

12. The method according to claim 10, wherein the microorganism belongs to a Corynebacterium sp.

13. The method according to claim 10, wherein the endogenous gluconate kinase activity is weakened by whole or partial deletion, partial substitution or insertion on a chromosomal gene encoding gluconate kinase.

14. The method according to claim 10, wherein the endogenous gluconate kinase activity is weakened by whole or partial deletion, partial substitution or insertion on a regulatory element for a chromosomal gene encoding gluconate kinase.

15. The method according to claim 10, wherein when there are two or more chromosomal genes coding proteins having gluconate kinase activity and having different amino acid sequences to each other, the endogenous gluconate kinase activity is weakened by whole or partial deletion, substitution or insertion on at least one of the chromosomal genes; or whole or partial deletion, partial substitution or insertion on a regulatory element for at least one of the chromosomal genes.

16. The method according to claim 10, wherein the microorganism is derived from Corynebacterium glutamicum KFCC10881.

17. The method according to claim 10, wherein the microorganism is identified as Corynebacterium glutamicum CA01-0892 deposited under accession No. KCCM 11085P.
